(19) United States
(12) Patent Application Publication
(10) Pub. No.: US 2022/0168524 A1
(43) Pub. Date: Jun. 2, 2022

(54) Breathing Apparatus Monitoring System

(71) Applicant: Loewenstein Medical Technology S.A., Luxembourg (LU)

(72) Inventors: Peter Kremeier, Karlsruhe (DE); Thomas Marx, Hamburg (DE)

(21) Appl. No.: 17/539,081

(22) Filed: Nov. 30, 2021

(30) Foreign Application Priority Data
Dec. 1, 2020 (DE) 102020131836.7

(51) Int. Cl. A61M 16/00

(52) U.S. Cl. CPC A61M 16/022; A61M 16/0003; A61M 2016/0027; A61M 2230/432; A61M 2016/003; A61M 2230/04

(57) ABSTRACT

A ventilator (1) with a respiratory device (2) for generating a respiratory gas flow for a ventilation and with a monitoring device (3) for monitoring a characteristic parameter (200) of the respiratory gas flow. A control device (4) is provided here and is suitable and configured to carry out a detection mode for a cardiac activity and to register for this a temporal profile (201) of the parameter (200) of the respiratory gas flow and to examine the temporal profile (201) of the parameter (200) for a profile structure feature (202) and to detect heartbeats in that the profile structure feature (202) fulfils a stored condition for a profile structure feature (202) which is caused by heartbeat.

### BACKGROUND

Technical Field: The present invention relates to a ventilator with at least one respiratory device for generating a respiratory gas flow for a ventilation and with at least one monitoring device for monitoring at least one characteristic parameter of the respiratory gas flow.

Such ventilators can sometimes be used for ventilation during a heart-lung resuscitation (so-called cardiopulmonary reanimation; in English: cardiopulmonary resuscitation, CPR). Through the taking over of the ventilation by means of equipment, a doctor or respectively aider is considerably relieved of a burden, so that a better concentration on the cardiac massage (CM) and other measures is possible.

In the prior art, ventilators have become known which not only provide the ventilation in a CPR, but can also assist in the carrying out of the CM. For this, the ventilator monitors by means of sensors changes is pressure and flow in the patient's lung during the CM. Through evaluation of the sensor signals, the apparatus detects how often and how intensively the aider performs a stroke for the CM. For example, after detecting 30 strokes, the apparatus can request an interrupting of the CM so that two ventilation strokes can take place by the apparatus or the aider. A detection of the (incipient) cardiac activity takes place here by means of an additional ECG.

### SUMMARY

In contrast, the object of the present invention is to improve the technical devices for assisting a CPR. In particular, a device is to be made available which can be used within a CPR and, in so doing, provides reliable and helpful information concerning the patient or respectively the success of the CPR, in particular without requiring an additional ECG.

This problem is solved by a ventilator of claim 1 and by a ventilator of claim 19 and by a monitoring system according to claim 20. Further developments and advantageous embodiments are the subject of the sub-claims. Further advantages and features will emerge from the general description and from the description of the example embodiments.

The ventilator according to the invention comprises at least one respiratory device for generating an (in particular controllable or respectively regulatable) respiratory gas flow for a ventilation. The ventilator comprises at least one monitoring device for monitoring (in particular for recording) at least one characteristic parameter of the respiratory gas flow. The ventilator comprises at least one control device (operatively connected to the monitoring device). The control device is suitable and configured here to carry out (at least intermittently) at least a detection mode for detecting a cardiac activity. The control device is suitable and configured for registering a temporal profile of the parameter of the respiratory gas flow by means of the monitoring device and for examining the registered temporal profile of the parameter for at least one profile structure feature. The control device is suitable and configured to detect heartbeats at least in that the profile structure feature at least partially fulfils, and in particular fulfils, at least one saved condition for a profile structure feature which is caused by heartbeat or respectively cardiogenic.

The present invention offers several advantages. The detection mode for detecting heartbeats on the basis of an evaluation of the recorded parameter offers a considerable advantage. This enables a technical heartbeat detection which is able to be implemented in a non-laborious manner and which is at the same time particularly reliable and automated. The apparatus or respectively system can thus detect heartbeats and can optionally inform the aider whether and when the patient's heart has begun to beat again. Accordingly, the aider is informed whether the CPR or respectively CM must continue or not.

In a CPR, it is indeed very crucial to detect the re-starting cardiac activity as early as possible, in order to be able to react thereto accordingly and to initiate further important measures. Hitherto, the detection of the re-starting cardiac activity was often very difficult, because the aider's concentration is directed to the CPR and in particular to the CM. With the present invention, the aider can now concentrate entirely on the necessary measures without having to continuously check whether the heart is beating again.

The technical or respectively constructional implementation of the invention is a particular advantage here, as the heartbeat detection can thereby be made available in an economically and medically meaningful manner. As the heartbeats are detected through the corresponding evaluation of a parameter, which is often recorded in any case in the context of the ventilation, a retrofitting of already existing apparatus is possible in a particularly simple manner, for example.

Preferably, the at least one parameter is a measurement for a flow of the respiratory gas and/or a measurement for a pressure of the respiratory gas and/or a measurement for a volume of the respiratory gas. In particular, the parameter is the respiratory gas flow and/or the respiratory gas pressure. It is possible that at least two parameters are registered, comprising at least the respiratory gas flow and the respiratory gas pressure. In the present technical field, the (respiratory gas) flow is frequently also designed as “flow”. Therefore the terms (respiratory gas) flow and flow can be used synonymously within the present invention. The (respiratory gas) flow is, in particular, a volume flow. In the sense of the invention, the respiratory gas is, in particular, such a respiratory gas which is delivered to the patient from the ventilator and/or a respiratory gas which escapes from the patient's lung and/or generally a respiratory gas which enables the detection of the heartbeat signals according to the invention.

Preferably, the at least one profile structure feature is a temporal change of the parameter. The profile structure feature is preferably a temporal flow change and/or a temporal pressure change or a measurement for one such. In particular, at least one measurement for a similarity to a temporal change of the parameter caused by heartbeats is provided as a condition. In particular, the profile structure feature describes how quickly and/or intensively and/or frequently the parameter changes over time. The drawing upon such parameters offers a detection of heartbeats which is particularly reliable and, at the same time, is able to be implemented in a technically particularly non-laborious manner.

Alternatively or additionally, the at least one profile structure feature can describe a geometric structure of the temporal profile of the parameter. As a condition, in particular at least one measurement is then provided for a similarity to a stored geometric structure, caused by heartbeats, of the temporal profile of the parameter. Such structures are, for example, symmetry characteristics, increases, turning points, zero points, high points, low points. Other features which are able to be identified within a function analysis or respectively curve sketching of the temporal profile of the parameter can also be drawn upon. The condition is then in particular a similarity of such a feature to at least one stored feature.

In a preferred and advantageous further development, the at least one profile structure feature describes a temporal flow change and/or a temporal pressure change or is one such. In particular, the condition is then at least that the temporal flow change and/or pressure change has a defined similarity to a temporal flow change and/or pressure change caused by heartbeats. As specific flow changes and pressure changes occur very reliably together with heartbeats, such a configuration offers a particularly reproducible detection of heartbeats.

Preferably as a condition it is at least predetermined how frequently and/or regularly the at least one profile structure feature occurs in the temporal profile of the parameter. Thus, for example, changes of the parameter caused by a noise can be discriminated with respect to cardiogenic changes.

The at least one profile structure feature describes in particular an occurrence of (local) maxima and/or (local) minima in the temporal profile of the parameter or is at least one such. In particular at least one maximum threshold for a value and/or amount of the parameter is then provided as a condition at a maximum or minimum. Such a configuration is particularly advantageous because in the case of heartbeats significant changes indeed occur which adopt maxima or respectively minima, but generally only small values.

In particular, in addition, however, a minimum threshold is also provided for the value and/or amount of the parameter at a maximum and/or minimum. Irrelevant changes or respectively a noise can thereby be dismissed. It is possible that in addition at least one filter is provided for the data.

Alternatively or additionally, the at least one profile structure feature can describe at least one of the following features in the temporal profile of the parameter: frequency of the maxima and/or minima per unit of time (frequency); number of the maxima and/or minima in a period of time; temporal regularity of the occurrence; (geometric) profile form of the maxima and/or minima. In particular the control device is suitable and configured to detect the cardiac activity at least depending on which frequency and/or which value and/or which geometric profile the maxima and/or minima have.

It is also possible and preferred that the at least one profile structure feature describes a maximum and/or minimum flow and/or a maximum and/or minimum pressure or is such a one. As a condition, preferably then at least one upper threshold is provided for an amount of the flow and/or pressure. Thereby, the specific but correspondingly weak flow- and/or pressure changes can be reliably detected. At a maximum of the pressure in particular a positive pressure or respectively a maximum overpressure is present. At a minimum of the pressure in particular a negative pressure or respectively a maximum underpressure is present. At a maximum of the flow, in particular a positive flow or respectively a respiratory gas flow flowing towards the patient or respectively in the direction of the patient's lung is present. At a minimum of the flow, in particular a negative flow or respectively a respiratory gas flow flowing in an opposing manner is present.

In a particularly preferred and advantageous embodiment, as a condition at least one upper threshold is provided for an amount of a flow of the respiratory gas. The upper threshold lies in particular between 0.01 l/s and 0.3 l/s and preferably between 0.02 l/s and 0.15 l/s. In particular as a condition provision is at least made that the maximum of the flow is not greater than 0.3 l/s. In particular as a condition provision is at least made that the minimum of the flow is not smaller than −0.02 l/s. Additionally or alternatively, as a condition an upper threshold can also be provided for the amount of another parameter and preferably of the pressure of the respiratory gas. The flow changes which are specific for heartbeats can thus be identified particularly well.

It is possible that as a condition provision is at least made that between a maximum and a minimum in the temporal profile of the parameter at least one sign change takes place. In particular at least one sign change and preferably only one sign change takes place between a maximum and a minimum in the temporal profile of the flow and/or of the pressure of the respiratory gas. Such a sign change is caused in particular by the contracting and expanding of the beating heart and its effect on the lung or respectively the thoracic cavity.

It is also possible that as a condition provision is at least made that temporal changes of the parameter and preferably an occurrence of maxima and/or minima in the temporal profile of the parameter take place with a defined frequency and/or regularity in the temporal profile of the parameter. The frequency describes in particular the number of changes within a defined time span and can also be designated as frequency. The regularity describes in particular the temporal intervals between the changes and preferably between the maxima or respectively minima.

As a condition, provision can also at least be provided that temporal changes of the parameter and preferably an occurrence of maxima and/or minima take place in the temporal profile of the parameter with a frequency of at least ten per minute and preferably 20 per minute and in particular in the range of 30 to 200 per minute. In particular, the frequency corresponds at least to a very low heart rate. In particular, the frequency, for example owing to the administered resuscitation drugs, is a maximum of 180 per minute. In particular, the maximum frequency corresponds to a high heart rate. Provision can be made that the control device establishes the frequency in that the patient's age is enquired. Thus, for younger people and in particular babies, a higher (maximum and/or minimum) frequency can be set than for older people.

In an advantageous further development, the control device is suitable and configured to examine the temporal profile of the parameter for at least one stored pattern caused by heartbeat. In particular, the control device is suitable and configured to detect heartbeats at least in that the pattern at least approximately occurs in the temporal profile. In particular, the pattern is defined by repetitions at least of one profile structure feature and preferably of at least two profile structure features. Particularly preferably, the pattern describes recurrent geometric structures of the temporal profile of the parameter. Particularly preferably, the pattern defines a temporal and/or geometric regularity of flow changes and/or pressure changes in the temporal profile of the parameter. The condition is then in particular a similarity of the temporal profile of the parameter to at least one stored pattern.

It is possible and advantageous that the control device is suitable and configured to determine a frequency of the at least one profile structure feature in the temporal profile of the parameter and, from the frequency, to determine a number of heartbeats and/or a heart rate. The number of heartbeats and/or the heart rate can also be determined from the pattern. The cardiac activity can thus be estimated better, after it has started again.

In a preferred and advantageous further development, the control device is suitable and configured to carry out the detection of the heartbeats (also) taking into consideration whether the at least one profile structure feature has occurred with a minimum quality and/or minimum number in the registered temporal profile of the parameter. The minimum number concerns in particular the number of maxima and/or minima. It is also possible to carry out the detection of the heartbeats taking into consideration whether the temporal profile of the parameter of the respiratory gas flow was registered during a minimum duration and/or whether the temporal profile of the parameter of the respiratory gas flow was registered with a defined minimum signal quality. The minimum quality defines in particular whether the profile structure feature as such is able to be detected and evaluated. In particular, the control device is suitable and configured to reject and/or to weight or respectively to verify the result of the detection of the heartbeats depending on the previously mentioned requirements.

In particular, the minimum duration is at least 2 or 3 seconds and preferably at least 5 seconds and particularly preferably at least 10 seconds.

In a particularly advantageous configuration, a carbon dioxide content of the blood and/or of the respiratory gas and/or a blood pressure and/or an oxygen concentration of the blood is able to be recorded as at least one further parameter by means of the monitoring device. Preferably, the control device is suitable and configured to register a temporal profile of the at least one further parameter and to take it into consideration for the detection of the heartbeats. Preferably, the control device is suitable and configured to carry out a plausibility check, taking into consideration the further parameter, in order to verify the result of the detection of the cardiac activity or respectively the detected heartbeats. The examination of the at least one further parameter preferably takes place as was previously described for the parameter. In particular, for the detection of heartbeats, a weighted taking into consideration of the parameter and of the further parameter takes place. The carbon dioxide content of the blood is described here in particular by a carbon dioxide partial pressure. In particular, the monitoring device has sensor means which are suitable for this, and/or is operatively connected to at least one external sensor means. The monitoring device preferably has at least one sensor means for the detection of the carbon dioxide content of the respiratory gas. Preferably, the incipient cardiac activity is detected through an increasing carbon dioxide content of the blood. The carbon dioxide content of the blood would drop again in the further process.

In all embodiments it is particularly preferred that the further parameter is a plethysmogram wave recorded in particular by means of pulse oximetry and/or an oxygen concentration recorded or respectively measured by means of pulse oximetry. In particular, the monitoring device is suitable and configured to record the further parameter by means of a pulse oximetry or respectively pulse oximetrically. For this, the monitoring device can be operatively connected to at least one pulse oximeter or can have a pulse oximeter itself.

In a particularly advantageous embodiment, by means of the monitoring device a plethysmogram wave, measured by means of pulse oximetry, and/or an oxygen saturation measured by means of pulse oximetry, is able to be recorded as at least one further parameter. Preferably, the control device is suitable and configured to register a temporal profile of the at least one further parameter and to at least partially take it into account for the detection of the heartbeats. The examination of the at least one further parameter preferably takes place as was previously described for the parameter. In particular, a weighted taking into consideration of the parameter and of the further parameter takes place for the detection of heartbeats. In particular, the monitoring device has a sensor means, suitable for this, and/or is operatively connected to at least one external sensor means. According to the invention, the incipient cardiac activity is detected by an increasing oxygen saturation and/or by a recurring plethysmogram wave.

Provision can be made that the control device is suitable and configured, for monitoring a cardiac massage, to register a temporal profile of the parameter and preferably of a flow and/or pressure of the respiratory gas at least during the cardiac massage and to evaluate an effect of the cardiac massage as a function of the temporal profile having a defined temporal flow change and/or pressure change. For this, in particular the quality (in particular the exerted pressure and/or the path of the stroke) and/or the quantity (in particular the number of strokes) is evaluated.

The quality is preferably determined by means of the amplitude of the pressures, recorded by sensor by the control unit, and/or flows. The quantity is preferably determined by means of the frequency of the pressures and/or flows recorded by sensor by the control unit.

In all embodiments it is particularly preferred that the control device is suitable and configured to issue, in the detection mode or respectively for a detection of the heartbeats, at least an indication that a cardiac massage is to be suspended and/or begun again. Preferably at the start of the detection mode or respectively before carrying out the detection of the heartbeats, an indication is issued that the cardiac massage is to be suspended. If the cardiac massage is continued, preferably at least one further indication takes place and/or the result of the detection is rejected. In particular at the end of the detection node or respectively after a carrying out of the detection of the heartbeats, an indication is issued that the cardiac massage is to be started again. In particular, the suspending and/or continuing of the cardiac massage is monitored by means of the monitoring device.

In particular, an acoustic and/or visual and/or haptic indication is issued. For example, an audible signal with a text indication can take place. It is also preferable that at least one speech output takes place, which states that the cardiac massage is to be suspended or respectively begun again.

It is also possible and advantageous that in the detection mode or respectively for a detection of the heartbeats at least one indication is issued that a ventilation is to be suspended and/or begun again. The control device can be suitable and configured to at least temporarily interrupt the ventilation in the detection mode. It is also possible that the ventilation in the detection mode is at least temporarily continued. The suspending can take place depending on the signal quality. For example, the ventilation is interrupted when particularly weak pressure—and/or flow changes must be recorded or the pressure—and/or flow signals are not clear and e.g. are too small or are noisy.

Preferably, the heartbeat detection can also take place during the ventilation or during the CM. The control device then detects in particular also the pressure- and/or flow signals in the respiratory gas which are generated by the (incipient) cardiac activity.

Preferably, the control device is suitable and configured to predetermine at least one acoustic and/or visual and/or haptic support indication for a cardiac massage and preferably for the rhythm thereof.

In particular, the control device is suitable and configured, as a function of a result of the detection of the heartbeats, to issue at least one indication and preferably to signal a presence of heartbeats acoustically and/or visually and/or haptically. Thus, even under difficult conditions, it can be perceived that the heart is beating again.

Particularly preferably, the respiratory device is able to be operated by means of the control device in at least one operating mode for the use of the ventilator in combination with a (separately carried out) cardiac massage. In such an operating mode (designated here as CPR operating mode), the respiratory device provides here at least a specific ventilation for a cardiopulmonary resuscitation (CPR). Such an embodiment is particularly advantageous, because in addition to the monitoring of the cardiac activity, a further support for the aider takes place in that the ventilation is also undertaken by the ventilator. For such a ventilation, the respiratory device is actuated by the control device preferably taking the parameter into consideration. The recording and evaluation of the parameter takes place here preferably as previously described. In particular, the ventilation takes place as a function of the cardiac massage. For example, after 30 strokes of the cardiac massage, two ventilation strokes take place by the respiratory device.

The control device is preferably suitable and configured to suspend (in a targeted manner) the ventilation of the CPR operating mode during the detection mode and preferably to continue the ventilation of the CPR operating mode again (in a targeted manner) after the detection mode as a function of the result of the detection. In particular, the control device is suitable and configured to carry out an automatic and/or adaptive changeover between detection mode and CPR operating mode. In particular, the result of the detection of the heartbeats is rejected when a ventilation and/or respiratory activity is present during the detection. However, it is also possible that the ventilation of the CPR operating mode is at least partially continued during the detection mode. For this, for example, an adapted pressure and/or flow can be provided for the respiratory device. It is also possible that the CPR operating mode and the detection mode are active in a parallel manner at least at times. With the suspending of the ventilation, preferably also the previously described indication that the cardiac massage is to be suspended is also issued.

The monitoring device is preferably suitable and configured to record the parameter also during the CPR operating mode and in particular during the carrying out of a CM. The control device is preferably suitable and configured to also register and examine the parameter during the CPR operating mode and in particular during the carrying out of a CM.

Preferably, as a condition provision is at least made that a temporal change of the parameter, preferably flow change and/or pressure change, has at least an amplitude the extent of which is at a maximum half, preferably at a maximum a quarter, the extent of at least an amplitude which the temporal change of the parameter has during the carrying out of a CM. In particular, the amplitude during the carrying out of a CM is at least twice as great as the amplitude during a heartbeat. In particular, as a condition provision is at least made that such an amplitude occurs within a defined period of time and/or with a defined frequency and/or regularity. In such an embodiment, in particular temporal changes of the parameter and e.g. amplitude are meant which are caused by the carrying out of the CPR, therefore by an externally exerted pressure on the thorax or respectively the heart.

Preferably as a condition provision is at least made that a temporal change of the parameter, preferably flow change and/or pressure change, has at least an amplitude the extent of which is at a maximum half, preferably at a maximum a third, of the extent at least of an amplitude which is provided or respectively set for the pressure and/or flow of the respiratory gas for the ventilation during the CPR operating mode. In particular, the amplitude during the ventilation within a CPR is at least three times as great as the amplitude during a heartbeat. In particular, as a condition provision is at least made that such amplitude occurs within a defined period of time and/or with a defined frequency and/or regularity.

In particular the control device is suitable and configured, for the monitoring of a cardiac massage, to register a temporal profile of a flow and/or pressure of the respiratory gas at least during the cardiac massage (CM) and to evaluate an effect of the cardiac massage as a function of the temporal profile having a defined temporal CM flow change and/or pressure change, wherein in addition flow changes and/or pressure changes are able to be recorded, which are caused by a beating heart and wherein the control device is suitable and configured to detect the heartbeat flow changes and/or pressure changes in a registered temporal profile of the flow and/or pressure as heartbeats. Preferably, the control device is suitable and configured to differentiate the CM flow change and/or pressure change from the heartbeat flow change and/or pressure change in that the CM flow change and/or pressure change have an amplitude which is at least twice as great as the heartbeat flow change and/or pressure change.

In particular, the control device is suitable and configured, for the monitoring of the ventilation, to register a temporal profile of a flow and/or pressure of the respiratory gas at least during the ventilation, wherein in addition flow changes and/or pressure changes are able to be recorded, which are caused by a beating heart and wherein the control device is suitable and configured to detect the heartbeat flow changes and/or pressure changes in a registered temporal profile of the flow and/or pressure as heartbeats. Preferably, the control device is suitable and configured to differentiate the ventilation flow change and/or pressure change from the heartbeat flow change and/or pressure change in that the ventilation flow change and/or pressure change have an at least three times as great amplitude as the heartbeat flow change and/or pressure change.

In an embodiment or in a ventilator according to the introductory clause of claim 1 it is preferred that in a or respectively in the detection mode, by means of the monitoring device, flow changes and/or pressure changes are able to be recorded which are caused by a beating heart in the case of an absent respiratory activity and that the control device is suitable and configured to detect the flow changes and/or pressure changes in a registered temporal profile of the flow and/or pressure as heartbeats. In particular, the control device detects the flow changes and/or pressure changes as heartbeats when the at least one condition is fulfilled. For the detection of the heartbeats, in particular the examination takes place of the temporal profile of the parameter for the at least one profile structure feature, as was previously described. The control device is suitable and configured in particular to detect heartbeats at least in that the profile structure feature is specific for cardiogenic flow oscillations and/or pressure oscillations.

The monitoring system according to the invention comprises at least one monitoring device for the monitoring at least of a characteristic parameter of a respiratory gas flow and at least one control device. Here, the control device is suitable and configured to carry out at least a detection mode for the detecting of a cardiac activity and to register for this a temporal profile of the parameter of the respiratory gas flow and to examine the temporal profile of the parameter for at least one profile structure feature and to detect heartbeats at least in that the profile structure feature at least partially fulfils at least a stored condition for a profile structure feature caused by heartbeat.

The monitoring system according to the invention solves the previously posed problem in a particularly advantageous manner. Here, for example, the monitoring system can be coupled to an already existing ventilator or can be integrated into one such, as was previously described. However, it is also possible and advantageous that the monitoring system is suitable and configured for an autonomous operation independently of a ventilator. The monitoring device and the control device are preferably configured as previously described for the ventilator. In particular, the monitoring system comprises at least a respiratory interface which is able to be connected to the patient. In particular, the respiratory interface is connected, with respect to flow, to the monitoring device via at least one tube.

In particular, the control device is suitable and configured to carry out the features formulated in a manner within the present description method. In particular, the control device is suitable and configured to examine the temporal profile for the profile structure features which are described here. The control device is suitable and configured in particular to compare a profile structure feature detected in the temporal profile with the at least one stored condition. For this, in particular at least one algorithm is stored in the control device. In particular, the control device comprises at least one electronic computer unit.

The saved condition corresponds in particular to a (previously empirically and/or theoretically determined) profile structure feature caused by heartbeat or is derived from one such. The definition of the profile structure feature caused by heartbeat takes place in particular in advance through detection of the temporal profile of the parameter of the respiratory gas flow in a patient with a beating heart and/or in another manner empirically and/or theoretically. Furthermore, this can take place through a relative increase of the measured end tidal CO₂ measurement or the recording of a plethysmogram wave recorded by pulse oximetry and/or the relative increase of the oxygen saturation measured by means of pulse oximetry. For the saved condition, such data are in particular prepared and, for example, abstracted.

Within the present invention, the terms cardiogenic and caused by heartbeat can preferably be used synonymously. Within the present invention, in addition to heartbeats, in particular also other typical types of cardiac activity are also understood under “caused by heartbeat”.

The monitoring device comprises in particular at least one sensor means for the recording of the parameter and preferably at least one flow sensor and/or at least one pressure sensor. The recorded parameter can be derived directly or indirectly from at least one sensor quantity.

The monitoring device comprises in particular at least one output device for the acoustic and/or visual and/or haptic issuing of information to a user. The output device comprises in particular at least one display and/or at least one loudspeaker. The output device can be provided at least partially by a touchscreen. The respiratory device comprises in particular at least one fan device and/or compressed gas source.

The control device is configured in particular for the at least intermittent actuation of the respiratory device taking the parameter into consideration. For example, thereby a predetermined or respectively adjustable respiratory gas pressure and/or respiratory gas flow can be set and preferably regulated. In particular, the respiratory device is able to be actuated not only taking into consideration the parameter, but also taking into consideration predetermined or respectively adjustable ventilation parameters by means of the control device. It is also possible that the control device is configured for the actuation, at least at times, of the respiratory device, taking into consideration detected heartbeats. For example, a ventilation can be paused when the heart has begun to beat again.

### BRIEF DESCRIPTION OF THE DRAWINGS

Further advantages and features of the present invention will emerge from the description of the example embodiments, which are explained below with reference to the enclosed figures.

In the figures there are shown:

FIG. 1 a purely schematic illustration of a monitoring system according to the invention, in a perspective view;

FIG. 2 a highly schematized graph of a temporal profile of a parameter to illustrate the detection of heartbeats by means of the invention; and

FIG. 3 a highly schematized graph of a temporal profile of a further parameter to illustrate the detection of heartbeats by means of the invention.

### DETAILED DESCRIPTION OF EXEMPLARY EMBODIMENT(S)

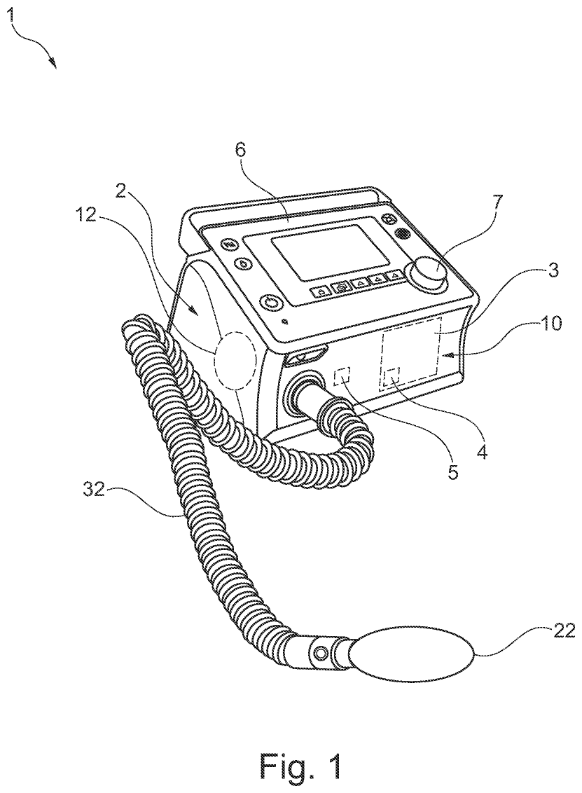

FIG. 1 shows a ventilator 1 according to the invention, which is equipped with a monitoring system 10 with a monitoring device 3. The monitoring system 10 can alternatively also be used as a separate device outside the ventilator 1.

The ventilator 1 has in the interior of its housing here a respiratory device 2 which is equipped with a fan device 12 for generating a respiratory gas flow. The respiratory gas flow is delivered to the patient via a tube device 32, coupled to the respiratory device 2, with a breathing mask 22. Alternatively to the breathing mask 22, other patient interfaces can also be used. Additionally or alternatively to the fan device 12, a compressed gas source can also be provided.

The monitoring device 3 serves for the monitoring of characteristic parameters of the respiratory gas flow. The respiratory device 2 is then actuated by means of a control device 4, taking into consideration the parameter and, if applicable, further ventilation parameters.

The ventilator 1 comprises here an output device 6 with an indicator or respectively a display and an operating device 7. Combinations of operating device 7 and output device 6 can also be provided here, for example in the manner of a touch-sensitive display surface or respectively a touchscreen. The output device 6 serves here also for the issuing of indications or respectively signals within a detection mode, described later, or respectively CPR operating mode. However, the output device 6 can issue its information or respectively indications on further display devices which are not shown here, e.g. on a computer display or a tablet or smartphone or suchlike.

The respiratory device 2 is operatively connected here to a sensor means 5 which has several sensors for the recording of the characteristic parameters of the respiratory gas flow and, if applicable, further characteristic values for the ventilation. For example, the sensor means 5 comprises a pressure sensor, not shown here in closer detail, which records the pressure conditions of the respiratory gas flow, and a flow sensor, likewise not shown in closer detail, which records the flow conditions of the respiratory gas flow.

The sensor means 5 is operatively connected to the monitoring device 3 and to the control device 4, so that the recorded values can be at least partially processed by these. The sensor means 5 can be arranged in the ventilator 1 or in the tube or in the patient interface.

The fan device 12 is actuated by the control device 4, which is arranged in a non-visible manner here within the housing, so that e.g. a CPAP or an APAP- or bi-level ventilation can be carried out. For a ventilation, the respiratory device 2 is set e.g. to a defined respiratory gas flow and/or a respiratory gas pressure. The control device 4 can provide a necessary minimum pressure and/or can compensate pressure fluctuations which are caused by the respiratory activity of the user. For example, the monitoring device 3 records the present pressure in the patient interface 22 by means of the sensor means 5 and readjusts the output of the fan device 12 accordingly, until a desired ventilation pressure is present.

In a detection mode, the ventilator 1 can be operated for a cardiac activity. An automatic heartbeat detection takes place here in the detection mode. The ventilator 1 can thus establish, e.g. within a CPR, whether and when the heart begins to beat again.

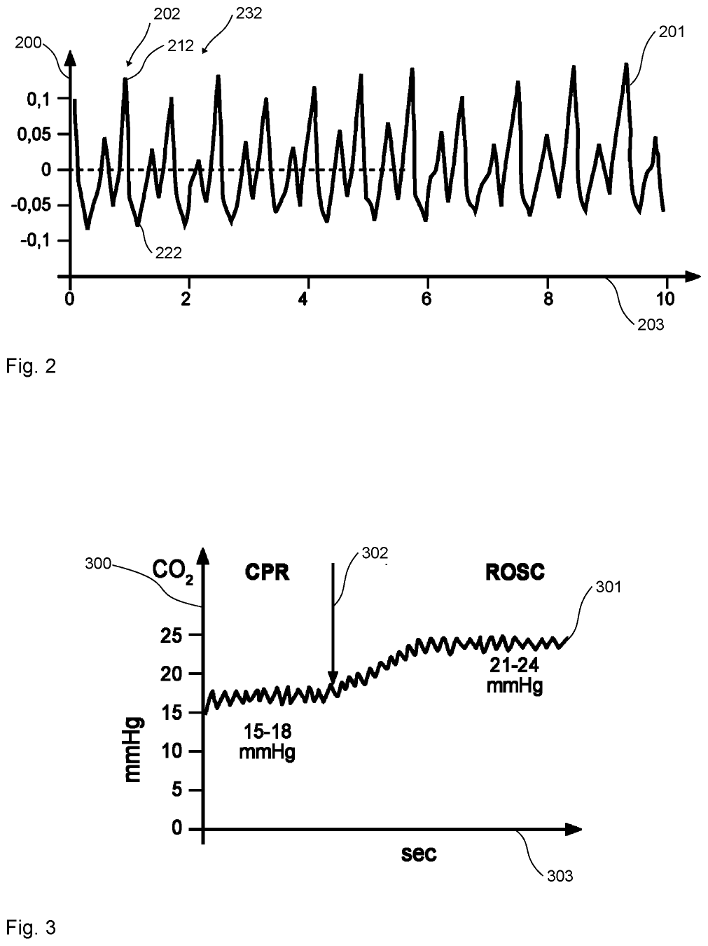

An example sequence of the detection mode is now described with reference to FIG. 2. In the detection mode, the control device 4 evaluates a temporal profile 201 of the parameter 200 of the respiratory flow, which the monitoring device 3 has previously recorded by sensor (as pressure or flow or volume) and which was registered by the control device 4. The profile 201 corresponds here to a representation of the parameter 200 over the time 203, which is indicated in seconds. The profile 201 which is shown here shows a 10-second window of a recording of the flow.

The control device 4 then examines the temporal profile 201 for one or more profile structure features 202. When the examined profile structure features 202 then fulfil at least one condition which is stored in the control device 4, and for example correspond to or are similar to stored heartbeat-specific profile structure features 202, the control device 4 evaluates this as the presence of a heartbeat.

In the example shown here, the parameter corresponds to a flow of the respiratory gas flow. A temporal profile 201 of the flow is therefore drawn upon for the detection of heartbeats. The flow is indicated here in litres per second. For the evaluation, the control device 4 examines the profile 201 for specific flow changes as profile structure feature 202, which are caused by the beating heart. Alternatively or additionally, as a parameter, a pressure of the respiratory gas flow can also be recorded and analysed in a corresponding manner.

In the example which is shown here, maxima 212 and minima 222 occurring over the time 203 are drawn upon as profile structure features 202. Thereby, the flow changes which are specifically caused by the beating heart can be detected in a particularly reliable manner. A superimposing or a comparison of the profile 201 which is shown here with an electrocardiogram (ECG) (not shown), clarifies the correlation between the pressure changes and the heartbeats particularly demonstratively. The ECG shows only the electrical activity of the heart, whereas the present invention enables it to also record the pumping activity of the heart. It can be readily seen here that a maximum amount of the flow corresponds to approximately 0.1 Vs. Such a rather smaller peak flow is characteristic for the flow changes which are caused by heartbeats. The amplitude of the flow signals caused by heartbeat lies in the range 0.51/min to 3.51/min for example at 0.6 to 2.31/min. The amplitude of the pressure signals caused by heartbeat lies in the range 0.2 cm/H₂O to 3.0 cm/H₂O for example at 0.4 to 2.2 cm/H₂O.

Additionally or alternatively, the control device 4 can also carry out a pattern detection for the profile 201, in order to detect heartbeats. For this, the profile 201 is examined for the occurrence of a particular pattern 232. In the profile 201 which is represented here for example a pattern 232 can be detected particularly well, which is characterized by the regularly occurring geometric structures with associated maxima 212 and minima 222 and the characteristic increases lying therebetween. The specific small, rhythmic oscillations in the respiratory gas flow can thus be seen here particularly well in the pattern.

The previously described detection mode can also be carried out by the monitoring system 10 according to the invention without the ventilator 1. The monitoring system 10 comprises here the monitoring device 3 and the control device 4. In order to record the parameter 200 of the respiratory gas flow, the monitoring device 3 is coupled to the patient by a suitable respiratory interface.

The monitoring system 10 can then be used for example as an autonomous system during a CPR, in order to enable an automated detection of heartbeats. The ventilation during the CPR then takes place for example manually or by means of a separate ventilator which is not shown in further detail here. The monitoring system 10 can also be coupled to an already present ventilator 1 or can be integrated therein, in order to extend its functionality by the detection mode.

The ventilator 1 offers here an operating mode for the use of the ventilator 1 in combination with a (separately carried out) cardiac massage. In such a CPR operating mode, the respiratory device 2 provides here a specific ventilation for the CPR. The ventilation can thus be undertaken by the ventilator 1 and the aider can concentrate on the CM. For example, after 30 strokes of the cardiac massage, two ventilation strokes take place by the respiratory device 2.

An example use of the ventilator 1 within a CPR proceeds for example as is described below. Firstly, the patient is connected to the ventilator 1 as intended. The ventilator 1 is moved into the CPR operating mode, so that the respiratory device 2 provides a ventilation which is particularly suitable for the CPR. Parallel to the ventilation or in an alternating manner, the cardiac massage takes place by the aider.

The ventilator 1 can assist the aider here in the CM and, for example, can signal acoustically and visually the rhythm and number of the CM. Signals can also be emitted which are indicative for the quality of the CM and indicate, for example, whether the pressure onto the thorax is sufficient. For this, pressure changes and flow changes are detected and evaluated by the control device 4 from the signals which are recorded by means of the monitoring device 3. As the pressure on the thorax brings about characteristic pressure changes and flow changes in the patient's lung, this offers a reliable monitoring.

After a particular number of, for example, 30 strokes, the ventilator 1 invites the aider to interrupt the CM. A particular number of, for example, two ventilation strokes are now applied by the respiratory device 2. After the ventilation strokes or else parallel thereto, the control device 4 activates the detection mode for the detection of the cardiac activity. For this, the temporal profile 201 of the parameter 200 is evaluated, as described above. For example, cardiogenic pressure changes and/or flow changes are sought in the temporal profile 201 and, if these fulfil particular conditions, these are identified as heartbeats.

Provision can be made here that during the recording of the parameter for the temporal profile 201 which is to be examined, a ventilation by the respiratory device 2 takes place. However, provision can also be made that the ventilation is then suspended in a targeted manner, for example so as not to influence the recording of the parameter 200. This is advantageous when the measurement conditions are difficult or respectively the signal quality of the measurement data is critical. The monitoring device 3 or respectively sensors shown here and the control unit 4 are set up and configured to record the signals caused by heartbeat also during the ventilation and/or during the CM. The signals caused by heartbeat can then be detected as oscillating fluctuations of the corresponding signals in a representation of the flow- or pressure signals.

When the control device 4 does not detect any heartbeats, an indication is emitted for the aider that the CM is to be continued. The CM is now carried out again as previously described and is accompanied by the ventilator 1 until the next detection mode takes place and so on. When the control device 4 detects heartbeats, it then emits a corresponding indication to the aider.

A further example sequence of the detection mode is now described with reference to FIG. 3. In so doing, in addition to the sequence described with reference to FIG. 2, also a plausibility check takes place here, in order to verify the result of the heartbeat detection.

For this, the control device 5 evaluates a profile 301 of a further parameter 300 registered over the time 303. The further parameter 300 here is a carbon dioxide partial pressure of the respiratory gas, which the monitoring device 3 has recorded by its own or an external sensor means.

It has been found that at the point in time 302 at which the circulatory activity begins again, a significant rise in the carbon dioxide partial pressure in the respiratory gas also occurs. Therefore, the control device 4 checks here the profile 301 of the further parameter 300 for such a rise in the carbon dioxide partial pressure. When the rise is present, e.g. an indication can be emitted. When no rise takes place, e.g. an indication can be emitted as a warning, or the detection mode can be continued or the result can be rejected.

Alternatively or additionally, a pulse oximetrically recorded plethysmogram wave and/or a pulse oximetrically measured oxygen concentration of the blood can also be drawn upon as further parameter 300. The monitoring device 3 is then e.g. operatively connected to a pulse oximeter. It can thus be established, e.g. by means of a significant change of the oxygen concentration, whether the circulatory activity has started again.

### LIST OF REFERENCE NUMBERS

1 ventilator
2 respiratory device
3 monitoring device
4 control device
5 sensor means
6 output device
7 operating device
10 monitoring system
12 fan device
22 breathing mask
32 tube device
200 parameter
201 profile
202 profile structure feature
203 time
212 maximum
222 minimum
232 pattern
300 parameter
301 profile
302 point in time
303 time

1. A ventilator with at least one respiratory device for generating a respiratory gas flow for a ventilation and with at least one monitoring device for monitoring at least one characteristic parameter of the respiratory gas flow,
characterized by
at least one control device which is suitable and configured to carry out at least one detection mode for a cardiac activity and to register for this a temporal profile of the parameter of the respiratory gas flow and to examine the temporal profile of the parameter for at least one profile structure feature and to detect heartbeats at least in that the profile structure feature at least partially fulfils at least one stored condition for a profile structure feature caused by heartbeat.

2. The ventilator according to the preceding claim, wherein the at least one parameter is a measurement for a flow of the respiratory gas or a measurement for a pressure of the respiratory gas.

3. The ventilator according to claim 1, wherein the at least one profile structure feature describes a temporal change of the parameter and wherein as a condition at least one measurement is provided for a similarity to a temporal change of the parameter caused by heartbeats.

4. The ventilator according to claim 1, wherein the at least one profile structure feature describes a temporal flow change or pressure change and wherein the condition is at least that the temporal flow change or pressure change has a defined similarity to a temporal flow change or pressure change caused by heartbeats.

5. The ventilator according to claim 1, wherein as a condition it is at least predetermined how frequently or regularly the at least one profile structure feature occurs in the temporal profile of the parameter.

6. The ventilator according to claim 1, wherein the at least one profile structure feature describes an occurrence of maxima or minima in the temporal profile of the parameter and wherein as a condition at least one maximum threshold is provided for a value or amount of the parameter at a maximum or minimum.

7. The ventilator according to claim 1, wherein the at least one profile structure feature describes a maximum or minimum flow or a maximum or minimum pressure, and wherein as a condition at least one upper threshold is provided for an amount of the flow or pressure.

8. The ventilator according to claim 1, wherein as a condition at least one upper threshold is provided for an amount of a flow of the respiratory gas, which lies between 0.01 litres per second and 0.3 litres per second and preferably between 0.02 litres per second and 0.15 litres per second.

9. The ventilator according to claim 1, wherein as a condition provision is at least made that a sign change takes place between a maximum and a minimum in the temporal profile of the parameter.

10. The ventilator according to claim 1, wherein as a condition provision is at least made that temporal changes of the parameter and preferably an occurrence of maxima or minima take place in the temporal profile of the parameter with a defined frequency or regularity in the temporal profile of the parameter.

11. The ventilator according to claim 1, wherein as a condition provision is at least made that temporal changes of the parameter and preferably an occurrence of maxima or minima take place in the temporal profile of the parameter with a frequency of at least 10 per minute and preferably 20 per minute or in the range of 30 to 200 per minute.

12. The ventilator according to claim 1, wherein the control device is suitable and configured to examine the temporal profile of the parameter for at least one saved pattern caused by heartbeat and to detect heartbeats at least in that the pattern occurs at least approximately.

13. The ventilator according to claim 1, wherein the control device is suitable and configured to determine a frequency of the at least one profile structure feature in the temporal profile of the parameter and to determine from the frequency a number of heartbeats or a cardiac frequency.

14. The ventilator according to claim 1, wherein the control device is suitable and configured to carry out the detection of the heartbeats, taking into consideration whether the at least one profile structure feature with a minimum quality or minimum number has occurred in the registered temporal profile of the parameter or whether the temporal profile of the parameter of the respiratory gas flow was registered during a minimum duration or whether the temporal profile of the parameter of the respiratory gas flow was registered with a defined minimum signal quality.

15. The ventilator according to claim 1, wherein by means of the monitoring device a carbon dioxide content of the blood or of the respiratory gas or a blood pressure or an oxygen concentration of the blood are able to be recorded as at least one further parameter and wherein the control device is suitable and configured to register a temporal profile of the at least one further parameter and to take it at least partially into consideration for the detection of the heartbeats.

16. The ventilator according to claim 1, wherein the control device is suitable and configured, for the monitoring of a cardiac massage, to register a temporal profile of a flow or pressure of the respiratory gas at least during the cardiac massage and to evaluate an effect of the cardiac massage as a function of the temporal profile having a defined temporal flow change or pressure change.

17. The ventilator according to claim 1, wherein the control device is suitable and configured, for a detection of the heartbeats, to emit at least one indication that a cardiac massage is to be suspended or begun again.

18. The ventilator according to claim 1, wherein the respiratory device is able to be operated by means of the control device in at least one operating mode for the use of the ventilator in combination with a cardiac massage and wherein the respiratory device in the operating mode provides at least one specific ventilation for a cardiopulmonary reanimation (in English: cardiopulmonary resuscitation, CPR), so-called CPR operating mode).

19. A ventilator with at least one respiratory device for generating a respiratory gas flow for a ventilation and with at least one monitoring device for monitoring at least one characteristic parameter of the respiratory gas flow, 1, wherein in a detection mode, by means of the monitoring device flow changes or pressure changes are able to be recorded, which are caused by a beating heart in an absence of spontaneous respiratory activity and wherein a control device is suitable and configured to detect as heartbeats the flow changes or pressure changes in a registered temporal profile of the flow or pressure.

20. A monitoring system with at least one monitoring device for monitoring at least one characteristic parameter of a respiratory gas flow and with at least one control device,
characterized in that
the control device is suitable and configured to carry out at least one detection mode for the detecting of a cardiac activity and to register for this a temporal profile of the parameter of the respiratory gas flow and to examine the temporal profile of the parameter for at least one profile structure feature and to detect heartbeats at least in that the profile structure feature fulfils at least one stored condition for a profile structure feature which is caused by heartbeat.
